(19) United States
(12) Patent Application Publication
(10) Pub. No.: US 2011/0212052 A1
(43) Pub. Date: Sep. 1, 2011

(54) METHODS AND COMPOSITIONS OF TREATING AND PREVENTING AUTOIMMUNE DISEASES

(75) Inventor: Adam Kaplin, Baltimore, MD (US)

(73) Assignee: The Johns Hopkins University, Baltimore, MD (US)

(21) Appl. No.: 13/055,880

(22) PCT Filed: Jul. 24, 2009
(86) PCT No.: PCT/US2009/051618

§ 371 Date: May 17, 2011

Related U.S. Application Data
(60) Provisional application No. 61/083,607, filed on Jul. 25, 2008.

(51) Int. Cl. A61K 38/19; A61K 39/395; A61P 19/02; A61P 37/06; A61P 5/14; A61P 1/00; A61P 3/10

(52) U.S. Cl. 424/85.1; 424/173.1

(57) ABSTRACT

Provided are methods and compositions for treating and/or preventing an autoimmune diseases, including inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

### CROSS-REFERENCE TO RELATED APPLICATIONS

This application claims the benefit of U.S. provisional application Ser. No. 61/083,607, filed on Jul. 25, 2008, the disclosure of which is incorporated by reference herein in its entirety.

### BACKGROUND

Inflammatory bowel diseases are a group of inflammatory disorders that affect areas of the gastrointestinal tract. The two most prevalent inflammatory bowel diseases are Crohn's disease and ulcerative colitis.

Crohn's disease (also known as granulomatous colitis and regional enteritis) is an autoimmune disease that affects approximately 500,000 patients in North America and is associated with a generalized increase in standardized mortality rate of approximately 1.5-fold and an average life expectancy of 58 years.

Traditional treatments for Crohn's disease often include one or more surgical resections of the bowel, which can lead to a syndrome called “short gut syndrome,” where the remaining length of the bowel is insufficient to support life without lifetime intravenous feeding with total parenteral nutrition.

Thus, there exists a need for improved methods for treating or preventing autoimmune diseases, including inflammatory bowel disease such as Crohn's disease.

### SUMMARY OF THE INVENTION

In one embodiment, the invention relates to methods for treating or preventing an autoimmune disease comprising administering to a subject in need thereof an effective amount of cyclophosphamide and anti-thymocyte globulin, wherein the autoimmune disease is inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease, and wherein the subject's immune system is reconstituted from stem cells that are continuously present in the subject following cyclophosphamide administration.

In another embodiment, the invention relates to methods for treating or preventing an autoimmune disease comprising administering to a subject in need thereof an effective amount of cyclophosphamide, anti-thymocyte globulin, and glatiramer acetate, wherein the autoimmune disease is inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

In yet another embodiment, the invention relates to methods for treating or preventing an autoimmune disease, the method comprising: (a) administering to a subject in need thereof an effective amount of cyclophosphamide and anti-thymocyte globulin, and (b) reconstituting the subject's immune system using stem cells that are continuously present in the subject following cyclophosphamide administration, wherein the autoimmune disease is inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

In still another embodiment, the invention relates to methods for treating or preventing an autoimmune disease comprising administering to a subject in need thereof an effective amount of cyclophosphamide and anti-thymocyte globulin, wherein the autoimmune disease is inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease, and the method does not comprise transplanting bone marrow or stem cells in the subject.

In another embodiment, the invention relates to methods for treating or preventing an autoimmune disease comprising administering to a subject in need thereof an effective amount of cyclophosphamide and anti-thymocyte globulin and allowing the subject's immune system to endogenously reconstitute, wherein the autoimmune disease is inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

In other embodiments, the invention relates to compositions comprising, (a) an effective amount of cyclophosphamide and anti-thymocyte globulin; and (b) a pharmaceutically acceptable carrier or vehicle. In some embodiments, the compositions further comprise an effective amount of glatiramer acetate.

In some embodiments, the invention relates to kits for the treatment or prevention of autoimmune disease comprising one or more doses of a composition of the invention, including one or more doses of cyclophosphamide and one or more doses of anti-thymocyte globulin. In certain embodiments, the kits further comprise one or more doses of glatiramer acetate.

### DETAILED DESCRIPTION OF THE INVENTION

As used herein, an “effective amount” is an amount effective for treating or preventing an autoimmune disease, including, for example, inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

As used herein, the term “about” when used in conjunction with an immediately following numeric indication means the referenced numeric indication plus or minus up to 10% of that referenced numeric indication.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening.

Some embodiments of the invention relate to methods for treating or preventing autoimmune diseases by administering to a subject cyclophosphamide and anti-thymocyte globulin and wherein the subject's immune system is reconstituted entirely from stem cells that were continuously present in the subject following cyclophosphamide administration. The autoimmune diseases treated or prevented by the methods of the invention include, but are not limited to, inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, and/or host vs. graft disease. As used herein, the term “inflammatory bowel diseases” or “IBD” includes art-recognized forms of a group of related conditions. Several major forms of IBD are known, and Crohn's disease (regional bowel disease, e.g., inactive and active forms) and ulcerative colitis (e.g., inactive and active forms) are the most common of these disorders. In addition, the IBD encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In another embodiment, the invention relates to methods for treating or preventing autoimmune diseases by administering to a subject cyclophosphamide, anti-thymocyte globulin, and glatiramer acetate. The autoimmune diseases treated or prevented by the methods of the invention include, but are not limited to, inflammatory bowel disease, ulcerative colitis, Crohn's disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

In some embodiments, the invention relates to methods for treating or preventing autoimmune diseases by administering to a subject in need thereof an effective amount of cyclophosphamide and anti-thymocyte globulin and allowing the subject's immune system to endogenously reconstitute. In certain embodiments the autoimmune diseases are inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, and/or host vs. graft disease. In some embodiments, the subject's immune system endogenously reconstitutes following administration of cyclophosphamide. In still other embodiments, the subject's immune system endogenously reconstitutes following administration of anti-thymocyte globulin. As used herein, the phrase “endogenously reconstitute” includes reconstitution without exogenous stem cells (e.g. without the administration of a stem cell transplant).

Achieving Immune Lymphablation

In certain embodiments, cyclophosphamide can be administered at an amount effective to achieve immune lymphablation. Immune lymphablation is achieved when the subject's white blood cell (“WBC”) count is about zero to about 100. Methods for measuring WBC in a subject are well known to those skilled in the art.

In some embodiments, the cyclophosphamide is administered at an amount of about 10 to about 100, about 25 to about 75, about 35 to about 65, or about 45 to about 55. In some embodiments, the cyclophosphamide is administered at an amount of about 10, about 20, about 30, about 40, about 45, about 50, about 55, about 60, about 70, about 80, about 90, or about 100 mg/kg/day. In other embodiments, the cyclophosphamide is administered at an amount of about 50 mg/kg/day. In some embodiments, the cyclophosphamide is administered daily for a period of about 3 to about 6 days. In some embodiments the cyclophosphamide is administered daily for a period of about 3, about 4, about 5 or about 6 days. In other embodiments, the cyclophosphamide is administered daily for a period of about 4 days. In still other embodiments, the cyclophosphamide is administered daily for a period of about 4 days at an amount of about 50 mg/kg/day.

In some embodiments, the cyclophosphamide is administered in the form of a suspension or solution. In some embodiments, the cyclophosphamide solution comprises cyclophosphamide reconstituted from lyophilized cyclophosphamide. The lyophilized cyclophosphamide can be reconstituted, for example, in phosphate buffered saline (“PBS”), a saline solution, water, or any combination thereof. In some embodiments, the concentration of the cyclophosphamide in the solution is about 20 mg/mL. In one embodiment, the cyclophosphamide solution is administered intravenously.

Cyclophosphamide can be gonadotoxic, thus potentially reducing a subject's fertility or putting a female subject, who subsequently becomes pregnant, at high risk for spontaneous abortion, preterm labor, and/or delivery of low birth weight infants. Therefore, in some embodiments the subject is not pregnant or at risk of pregnancy. If the female subject is at risk for pregnancy, the methods can further comprise administration of a gonadotropin releasing hormone agonistic analog (“GnRH-a”) as described, for example, in Z. Blumenfeld, “Gender difference: fertility preservation in young women but not in men exposed to gonadotoxic chemotherapy.” Minerva Endocrinol., 32: 23-34 (2007), the contents of which are incorporated by reference herein in their entirety. In one embodiment, the GnRH-a is goserelin acetate, leuprolide acetate, or nafarelin acetate. In one embodiment, administration of the GnRH-a reduces any gonadotoxic effect of the cyclophosphamide. In some embodiments, the GnRH-a is administered concurrently with the cyclophosphamide. In other embodiments, the GnRH-a is administered monthly starting about 4 to 6 months before the first dose of cyclophosphamide, and continuing until the final administration of the cyclophosphamide. In some embodiments, the GnRH-a is administered monthly at an amount of about 3 to about 4 mg.

In some embodiments, the methods further comprise administering an effective amount of anti-thymocyte globulin (“ATG”) to the subject. In one embodiment, the ATG is administered concurrently with or after the administration of the cyclophosphamide. In another embodiment, the ATG reduces the number of the subject's T cells.

In some embodiments, the ATG is administered at an amount of about 1 to about 20 mg/kg/day. In other embodiments, the ATG is administered at an amount of about 10 to about 20 mg/kg/day. In other embodiments, the ATG is administered at an amount of about 1.5 to about 2.5 mg/kg/day. In other embodiments, the ATG is administered at an amount of about 2.5 mg/kg/day. In other embodiments, the ATG is administered at an amount of about 1.5 mg/kg/day. In some embodiments, the ATG is administered daily for a period of about 1 to about 14 days. In other embodiments, the ATG is administered daily for a period of about 3 to about 6 days. In other embodiments, the ATG is administered daily for a period of about 4 days. In still other embodiments, the ATG is administered daily for a period of about 4 days at an amount of about 2.5 mg/kg/day. In some embodiments, the ATG is administered intravenously.

In some embodiments, the ATG is administered concurrently with the administration of the cyclophosphamide. In some embodiments, each dose of ATG is administered on the same day that a dose of cyclophosphamide is administered. In other embodiments, the ATG is administered after the administration of the first or second dose of cyclophosphamide.

In other embodiments, ATG is administered after the administration of the cyclophosphamide. In some embodiments, the first dose of ATG is administered about 0 to about 6 days after the administration of the final dose of cyclophosphamide. In other embodiments, the ATG is administered about 6 days after the administration of the final dose of cyclophosphamide.

In other embodiments, the methods further comprise administering an effective amount of granulocyte colony stimulating factor (“GCSF”) to the subject. In one embodiment, the GCSF is administered at an amount that is effective to promote reconstitution of the immune system. Reconstitution of the immune system is achieved when the subject's absolute neutrophil count exceeds 1.0×10⁹ cells/L of blood. Techniques for determining absolute neutrophil count are well known to persons skilled in the art.

The GCSF can be administered prior to, subsequent to, or concurrently with the cyclophosphamide. In one embodiment, the GCSF is administered subsequent to when the cyclophosphamide achieves immune lymphablation. In some embodiments, the GCSF is administered to the subject about 2 to about 8 days after the administration of a dose of cyclophosphamide. In another embodiment, administration of cyclophosphamide is discontinued prior to administering GCSF. In some embodiments, the GCSF is administered to the subject about 2, about 3, about 4, about 5, about 6, about 7 or about 8 days after the administration of the final dose of cyclophosphamide. In one embodiment, the GCSF is administered intravenously or subcutaneously.

In some embodiments, the GCSF is administered at an amount of about 2 to about 10 μg/kg/day. In other embodiments, the GCSF is administered at an amount of about 5, about 6, about 7, about 8 or about 9, μg/kg/day. In some embodiments, the GCSF is administered to the subject until the subject's absolute neutrophil count exceeds 1.0×10⁹ cells/L of blood. In other embodiments, the GCSF is administered daily for a period of about 2 days.

In other embodiments, the methods further comprise administering an effective amount of one or more antibiotics to the subject. In one embodiment, the antibiotic is administered at an amount that is effective to minimize or prevent infection during reconstitution of the immune system. The antibiotic can be administered before, during or after the administration of the cyclophosphamide. Suitable antibiotics include, but are not limited to, norfloxacin, fluconaxole, valacyclovir, ciprofloxacin, metronidazole, clarithromycin, and levofloxacin.

Preventing Re-Activation of the Autoimmune Disease

In one aspect of the invention, glatiramer acetate can be administered at an amount effective to prevent re-activation of the autoimmune disease. Re-activation of the autoimmune disease, as used herein, includes the appearance of one or more clinical or pathological indicators of the autoimmune disease. Clinical or pathological indicators of Crohn's disease include, for example, diarrhea, gastrointestinal bleeding, and/or abdominal pain. Glatiramer acetate is also known as copolymer-1 (see U.S. Pat. Nos. 5,981,589; 6,054,430; 6,342,476; 6,362,161; 6,620,847; 6,939,539; and 7,199,098, each of which is incorporated by reference herein in its entirety). In one embodiment, the glatiramer acetate is in the form of a composition. In one embodiment, the composition is that which is sold under the trademark Copaxone®.

In some embodiments, the glatiramer acetate is administered at an amount of about 20 to about 40 mg/kg/day. In other embodiments, the glatiramer acetate is administered at an amount of about 20 mg/kg/day. In some embodiments, the glatiramer acetate is administered daily for a period of at least about 30 days. In some embodiments, the glatiramer acetate is administered daily for a period of about 30 days to about 1 year. In other embodiments, the glatiramer acetate is administered daily for at least about 1 month, 2 months, 3 months, 4 months, 5 months, 6 months, 7 months, 8 months, 9 months, 10 months or 11 months.

In some embodiments, the glatiramer acetate is administered before, concurrently with, or after the administration of the cyclophosphamide. In some embodiments, the first dose of glatiramer acetate is administered about 0 to about 30 days after the administration of the final dose of cyclophosphamide. In some embodiments, the first dose of glatiramer acetate is administered about 30 days after the administration of the final dose of cyclophosphamide. In other embodiments, the first dose of glatiramer acetate is administered about 0 to about 30 days, e.g., about 1, about 2, about 3, about 4, about 5, about 6, about 7, about 8, about 9, about 10, about 15, about 20, about 25, or about 30 days, before the administration of the first dose of cyclophosphamide. In some embodiments, the first dose of glatiramer acetate is administered about 1, about 2, about 3, about 4, about 5, about 6, about 7, about 8, about 9, about 10, about 15, about 20, about 25, or about 30 days after the administration of the final dose of cyclophosphamide. In some embodiments, the glatiramer acetate is administered subcutaneously.

The cyclophosphamide, ATG, and/or glatiramer acetate, and any combination thereof, in the above-described methods can conveniently be administered as a component of a composition that comprises a physiological carrier or vehicle. It will be appreciated that the present invention further includes combinations of the pharmaceutical compounds, dosages and administrations disclosed herein.

The compositions can be administered orally, by infusion, by bolus injection, by absorption through epithelial or mucocutaneous linings (e.g., oral, rectal, and intestinal mucosa), or by any other convenient route of administration. Administration can be systemic or local. Various delivery systems are known, e.g., encapsulation in liposomes, microparticles, microcapsules, capsules, and can be administered.

Methods of administration include, but are not limited to, intradermal, intramuscular, intraperitoneal, intravenous, ocular, subcutaneous, intranasal, epidural, oral, sublingual, intracerebral, intravaginal, transdermal, rectal, by inhalation, or topical, particularly to the ears, nose, eyes, or skin. In some instances, administration will result in the release of the cyclophosphamide, ATG, or glatiramer acetate into the bloodstream. The mode of administration can be left to the discretion of the practitioner.

In other embodiments, it can be desirable to administer the cyclophosphamide, ATG, or glatiramer acetate locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the cyclophosphamide, ATG, or glatiramer acetate into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the cyclophosphamide, ATG, or glatiramer acetate can be formulated as a suppository, with traditional binders and excipients such as triglycerides.

In another embodiment, the cyclophosphamide, ATG, or glatiramer acetate can be delivered in a vesicle, in particular a liposome (see Langer, Science 249:1527-1533 (1990) and Treat or prevent et al., Liposomes in the Therapy of Infectious Disease and Cancer 317-327 and 353-365 (1989), the contents of which are incorporated by reference herein in their entirety).

In yet another embodiment, the cyclophosphamide, ATG, or glatiramer acetate can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989), the contents of each of which are incorporated by reference herein in their entirety).

In yet another embodiment a controlled- or sustained-release system can be placed in proximity of a target of the cyclophosphamide, ATG, or glatiramer acetate, e.g., the spinal column, brain, skin, lung, thyroid gland, colon or gastrointestinal tract, thus requiring only a fraction of the systemic dose.

The compositions can optionally comprise a suitable amount of a pharmaceutically acceptable excipient so as to provide the form for proper administration to the subject. Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment the pharmaceutically acceptable excipients are sterile when administered to a subject. Water is a particularly useful excipient when the cyclophosphamide, ATG, or glatiramer acetate is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The compositions, if desired, can also contain minor amounts of wetting or emulsifying agents, or pH buffering agents.

The compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155, the contents of which are incorporated by reference herein in their entirety). Other examples of suitable pharmaceutical excipients are described in Remington's Pharmaceutical Sciences 1447-1676 (Alfonso R. Gennaro eds., 19th ed. 1995, the contents of which are incorporated by reference herein in their entirety).

In one embodiment the cyclophosphamide, ATG, or glatiramer acetate is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a cyclophosphamide, ATG, or glatiramer acetate is also suitable for orally administered compositions. In these latter platforms, fluid from the environment surrounding the capsule is imbibed by the driving compound, which swells to displace the agent or agent composition through an aperture. These delivery platforms can provide an essentially zero-order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the cyclophosphamide, ATG, or glatiramer acetate can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the cyclophosphamide, ATG, or glatiramer acetate is to be administered by infusion, they can be dispensed, for example, with an infusion bottle containing sterile pharmaceutical grade water or saline. Where the cyclophosphamide, ATG, or glatiramer acetate is administered by injection, an ampule of sterile water for injection or saline can be provided so that the ingredients can be mixed prior to administration.

The cyclophosphamide, ATG, or glatiramer acetate can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

The cyclophosphamide, ATG, and/or glatiramer acetate, and any combination thereof, can also be provided in the form of a kit to simplify the administration to the subject. A typical kit of comprises a unit dosage form of cyclophosphamide, ATG, or glatiramer acetate. In one embodiment the unit dosage form is within a container, which can be sterile, containing an effective amount of cyclophosphamide, ATG, or glatiramer acetate and a physiologically acceptable carrier or vehicle. The kit may comprise one or more doses of glatiramer acetate. The kit can further comprise a label or printed instructions instructing the use of a composition of the invention, including cyclophosphamide, ATG, or glatiramer acetate, and any combination thereof, to treat or prevent an autoimmune disease. The kits can further comprise a device that is useful for administering the unit dosage forms, as detailed herein. Examples of such a device include, but are not limited to, a syringe, a drip bag, a patch, an inhaler, and an enema bag.

Having described the invention with reference to certain embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Effect of Cyclophosphamide and Glatiramer Acetate (“GA”) on Crohn's Disease in Mice (TNBS Colitis Model)

a. Induction of Crohn's Colitis

Mice are randomized into treatment groups with the average body weight equivalent in each group. 6 to 12 week old Balb/c mice are treated with 2 mg of the hapten trinitrobenzene sulfonic acid (TNBS) in 0.1 ml of 50% ethanol by intrarectal administration using a 18 G feeding tube (Fine Science Tools, Foster City, Calif.). This treatment is repeated weekly for 5 weeks in order to induce Crohn's-like colitis.

After 5 weeks of treatment, the mice are weighed and observed for clinical signs of Crohn's-like colitis. Crohn's-like colitis is determined by weight loss, change in stool consistency, and/or microscopic blood in the stool by Hemoccult testing. Clinical signs of Crohn's-like colitis are assessed using the Daily Activity Index (“DAI”). The DAI is the average of change in weight (0, <1%; 1, 1-5%; 2, 5-10%, 23, 10-20% and 4, >20%), intestinal bleeding (0, negative; 2, microscopic blood; 4, visible blood), and stool consistency (0, normal; 2, loose stools; 4, diarrhea).

b. Administration of Cyclophosphamide and Glatiramer Acetate

After establishment of Crohn's-like colitis (day 0), one-half of the mice are administered cyclophosphamide via intraperitoneal or intravenous injection in phosphate-buffered saline (PBS) (20 mg/ml) at a dose of 100-200 mg/kg. On day 2 (±2), as determined by colitis progression), GA is administered subcutaneously at a dose of 500-2000 micrograms/mouse in PBS/mannitol for up to five consecutive days.

The other half of the mice are administered with mock injections (vehicle) or intrarectal 50% ethanol as a control.

For the intravenous injections, the mice are warmed with a heat lamp (approximately 18-25 inches from the cage floor) while in their cage for 3-5 minutes to dilate their blood vessels; they are then individually restrained in a cone or Broome-type restraining device (VWR catalogue number 10718-030) for the intravenous injection administered into the lateral tail vein with a 28-30 gauge needle.

c. Results

Two months after treatment as described in step b, the mice are weighed and observed for clinical signs of Crohn's-like colitis, as described in step a above.

### Example 2

### Effect of Cyclophosphamide and Glatiramer Acetate on Crohn's Disease in Mice (CD45RB^{High} Transfer Model)

a. Induction of Crohn's Colitis

CD4+ CD45RB^{high} and CD4+ CD45RB^{low} T-cell subsets are isolated from spleens of wild-type C57BL/6 female mice using immunomagnetic selection and 0.5×10⁶ cells are injected intraperitoneally to RAG −/−mice on the same background.

4 weeks after adoptive transfer, the mice are weighed and observed for clinical signs of Crohn's-like colitis. Crohn's-like colitis is determined by weight loss, change in stool consistency, and/or microscopic blood in the stool by Hemoccult testing. Clinical signs of Crohn's-like colitis are assessed using the Daily Activity Index (“DAI”). The DAI is the average of change in weight (0, <1%; 1, 1-5%; 2, 5-10%, 23, 10-20% and 4, >20%), intestinal bleeding (0, negative; 2, microscopic blood; 4, visible blood), and stool consistency (0, normal; 2, loose stools; 4, diarrhea).

b. Administration of Cyclophosphamide and Glatiramer Acetate

After establishment of Crohn's-like colitis (day 0), one half of the CD4+ CD45RB^{high} and one-half of the CD4+ CD45RB^{low} mice are administered cyclophosphamide via intraperitoneal or intravenous injection in phosphate-buffered saline (PBS) (20 mg/ml) at a dose of 100-200 mg/kg. On day 2 (±2), as determined by colitis progression), GA is administered subcutaneously at a dose of 500-2000 micrograms/mouse in PBS/mannitol for up to five consecutive days.

The other half of the CD4+ CD45RB^{high} and the CD4+ CD45RB^{low} mice are administered with mock injections (vehicle) as a control.

For the intravenous injections, the mice are warmed with a heat lamp (approximately 18-25 inches from the cage floor) while in their cage for 3-5 minutes to dilate their blood vessels; they are then individually restrained in a cone or Broome-type restraining device (VWR catalogue number 10718-030) for the intravenous injection administered into the lateral tail vein with a 28-30 gauge needle.

c. Results

Two months after treatment as described in step b, the mice are weighed and observed for clinical signs of Crohn's-like colitis, as described in step a above.

### Example 3

### Effect of Cyclophosphamide and Glatiramer Acetate on Crohn's Disease in Mice (C3H.IL10−/− Model)

a. Induction of Crohn's Colitis

Mice are randomized into treatment groups with the average body weight equivalent in each group. 6 to 12 week old C3H.IL10−/− mice are administered one oral gavage with 10⁸ LF82 E. coli bacteria to establish severe colitis.

After 2 months, the mice are weighed and observed for clinical signs of Crohn's-like colitis. Crohn's-like colitis is determined by weight loss, change in stool consistency, and/or microscopic blood in the stool by Hemoccult testing. Clinical signs of Crohn's-like colitis are assessed using the Daily Activity Index (“DAI”). The DAI is the average of change in weight (0, <1%; 1, 1-5%; 2, 5-10%, 23, 10-20% and 4, >20%), intestinal bleeding (0, negative; 2, microscopic blood; 4, visible blood), and stool consistency (0, normal; 2, loose stools; 4, diarrhea).

b. Administration of Cyclophosphamide and Glatiramer Acetate

After establishment of Crohn's-like colitis (day 0), one-half of the mice are administered cyclophosphamide via intraperitoneal or intravenous injection in phosphate-buffered saline (PBS) (20 mg/ml) at a dose of 100-200 mg/kg. On day 2 (±2), as determined by colitis progression), GA is administered subcutaneously at a dose of 500-2000 micrograms/mouse in PBS/mannitol for up to five consecutive days.

The other half of the mice are administered with mock injections (vehicle) or intrarectal 50% ethanol as a control.

For the intravenous injections, the mice are warmed with a heat lamp (approximately 18-25 inches from the cage floor) while in their cage for 3-5 minutes to dilate their blood vessels; they are then individually restrained in a cone or Broome-type restraining device (VWR catalogue number 10718-030) for the intravenous injection administered into the lateral tail vein with a 28-30 gauge needle.

c. Results

Two months after treatment as described in step b, the mice are weighed and observed for clinical signs of Crohn's-like colitis, as described in step a above.

### Example 4

### Effect of Cyclophosphamide and Glatiramer Acetate on Crohn's Disease in Humans

Ten human subjects are administered in an open-label format with cyclophosphamide and glatiramer acetate. Human subjects are offered entry into the study if they meet all of the inclusion criteria and none of the exclusion criteria.

a. Inclusion/Exclusion Criteria

Human subjects are male or female, aged 18-70 inclusive. Human subjects must have evidence of ongoing disease activity with evidence of active disease on ileocolonoscopy and a Crohn's Disease of Activity Index of greater than 250.

Exclusion criteria are (1) any risk of pregnancy, (2) cardiac ejection fraction of <45%, (3) serum creatinine >2.0, (4) human subjects who are pre-terminal or moribund, (5) bilirubin >2.0, transaminases>2× normal, (6) human subjects with CDAI less than 250, (7) human subjects with active infections until infection is resolved or adequately managed, and (8) human subjects with WBC count <3000 cells/μL, platelets <100,000 cells/μL and untransfused hemoglobin <10 g/dL.

b. Administration of Cyclophosphamide

Human subjects are administered with cyclophosphamide intravenously at a dose of 50 mg/kg/day on Day −3 to Day 0.

Adequate diuresis should be maintained before and following cyclophosphoramide administration to prevent hemorrhagic cystitis. Prophylaxis for cyclophosphamide-induced hemorrhagic cystitis (generally either MESNA (2-mercaptoethane sulfonate sodium) or forced diuresis) is directed according to established clinical practice guidelines used by the SCT (stem cell transplant) program.

On Day 6 (six days after the final dose of cyclophosphamide) all human subjects receive GCSF at a dose of 5 μg/kg/day until their absolute neutrophil count exceeds 1.0×10⁹ per liter for two consecutive days. Human subjects are also routinely given antibiotics (norfloxacin, fluconazole or valacyclovir) until their absolute neutrophil count exceeds 1.0×10⁹ per liter for two consecutive days.

c. Administration of Glatiramer Acetate

Human subjects are administered with glatiramer acetate subcutaneously at a dose of 20 mg/kg on Day 30 (30 days after the final dose of cyclophosphamide), and continue to receive daily doses of glatiramer acetate at 20 mg/kg indefinitely.

d. Evaluation of Results

Baseline clinical evaluations are conducted at months −3 and 0; treatment and follow-up visits at months 3, 6, 9, 12, 15, 18, 21 and 24 months. Colonoscopies are conducted at months −3, 3, 12, and 24 to monitor the course of the disease progression after treatment.

The present invention provides methods and composition for the treatment and/or prevention of autoimmune diseases. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference.

1. A method for treating or preventing an autoimmune disease, the method comprising administering to a subject in need thereof an effective amount of cyclophosphamide and anti-thymocyte globulin, wherein the autoimmune disease is inflammatory bowel disease, rheumatoid arthritis, diabetes mellitus, celiac disease, autoimmune thyroid disease, autoimmune liver disease, Addison's Disease, Sjogren's Syndrome, transplant rejection, graft vs. host disease, or host vs. graft disease;
and wherein the subject's immune system is reconstituted from stem cells that are continuously present in the subject following cyclophosphamide administration.

2. A method for treating or preventing an autoimmune disease, the method comprising administering to a subject in need thereof an effective amount of cyclophosphamide, anti-thymocyte globulin, and glatiramer acetate, wherein the autoimmune disease is inflammatory bowel disease, rheumatoid arthritis, diabetes mellitus, celiac disease, autoimmune thyroid disease, autoimmune liver disease, Addison's Disease, Sjogren's Syndrome, transplant rejection, graft vs. host disease, or host vs. graft disease.

3. (canceled)

4. The method of claim 1, wherein the method does not comprise transplanting bone marrow or stem cells in the subject.

5. (canceled)

6. The method of claim 1, wherein the subject's immune system endogenously reconstitutes following administration of cyclophosphamide.

7. (canceled)

8. The method of claim 1, wherein stem cells are not administered to the subject between cyclophosphamide administration and reconstitution of the subject's immune system.

9. The method of claim 1, wherein the autoimmune disease is inflammatory bowel disease.

10. The method of claim 9, wherein the inflammatory bowel disease is ulcerative colitis.

11. The method of claim 9, wherein the inflammatory bowel disease is Crohn's disease.

12. The method of claim 1, wherein the amount of cyclophosphamide is about 25 to about 75 mg/kg/day.

13. The method of claim 12, wherein the amount of cyclophosphamide is about 50 mg/kg/day.

14. The method of claim 1, wherein the cyclophosphamide is administered for about 3 to about 6 days.

15. (canceled)

16. The method of claim 1, wherein the amount of the anti-thymocyte globulin is effective to reduce the number of the subject's T cells.

17. The method of claim 1, wherein the amount of the anti-thymocyte globulin is about 1 to about 20 mg/kg/day.

18. The method of claim 1, wherein the amount of the anti-thymocyte globulin is about 1.5 to about 2.5 mg/kg/day.

19. (canceled)

20. The method of claim 1, wherein the cyclophosphamide and the anti-thymocyte globulin are administered concurrently.

21. The method of claim 1, wherein the anti-thymocyte globulin is administered subsequent to administering the cyclophosphamide.

22. The method of claim 1, wherein the anti-thymocyte globulin is administered subsequent to when the cyclophosphamide achieves immune lymphablation.

23. The method of claim 1, wherein the anti-thymocyte globulin is administered for about 3 to about 6 days.

24. (canceled)

25. The method of claim 1, further comprising administering an effective amount of granulocyte colony stimulating factor or an antibiotic.

26. (canceled)

27. The method of claim 1, further comprising administering an effective amount of glatiramer acetate.

28-29. (canceled)

30. The method of claim 27, wherein the glatiramer acetate is administered for about 30 days to about 1 year.

31. The method of claim 27, wherein the glatiramer acetate is administered before, concurrently with, or after the administration of the cyclophosphamide.

32. The method of claim 27, wherein the first dose of the glatiramer acetate is administered about 0 to about 30 days before the first dose of the cyclophosphamide.

33. The method of claim 27, wherein the first dose of the glatiramer acetate is administered about 0 to about 30 days after the final dose of the cyclophosphamide.

34. The method of claim 27, wherein the glatiramer acetate is administered for about 30 days to about 1 year.

35. A composition comprising (a) an effective amount of cyclophosphamide and anti-thymocyte globulin; and (b) a pharmaceutically acceptable carrier or vehicle.

36. The composition of claim 35, further comprising an effective amount of glatiramer acetate.

37. A kit for treating or preventing an autoimmune disease comprising (a) one or more doses of cyclophosphamide; and (b) one or more doses of anti-thymocyte globulin, wherein the autoimmune disease is inflammatory bowel disease, rheumatoid arthritis, diabetes mellitus, celiac disease, autoimmune thyroid disease, autoimmune liver disease, Addison's Disease, Sjögren's Syndrome, transplant rejection, graft vs. host disease, or host vs. graft disease.

38. The kit of claim 37, further comprising one or more doses of glatiramer acetate.
